(19) United States
(12) Patent Application Publication
(10) Pub. No.: US 2008/0105760 A1
(43) Pub. Date: May 8, 2008

(54) Suction-Mountable Aromatic Device and Method for Using the Same

(75) Inventors: Douglas M. Sheffield, Chesterfield, MO (US); Tamara K. Sheffield, Chesterfield, MO (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: We-Flex, LLC

(21) Appl. No.: 11/557,729

(22) Filed: Nov. 8, 2006

(51) Int. Cl. A61L 9/12; A61L 9/04

(52) U.S. Cl. 239/56; 239/54

(57) ABSTRACT

A deformable aromatic device mountable by suction on a mounting surface including a body including a flexible base formed from a first material. The flexible base has an outer surface, an inner surface including a concave portion, and a periphery around the body. The body further includes a message formed on the body. The device further includes a second material selected to emit a desired aroma. The body is deformable from an undeformed shape to an actuated shape to mount the body on the mounting surface by positioning the inner surface adjacent the mounting surface when the body is undeformed thereby forming a cavity between the concave portion and the mounting surface and deforming the body to its actuated shape thereby sealing the body against the mounting surface and forcing fluid out of the cavity to create suction between the body and the mounting surface.

### BACKGROUND OF THE INVENTION

The present invention relates to aromatic devices and, more particularly, to suction-mountable aromatic devices.

Aromatic devices include a material emitting a desired aroma. Depending on the physical and/or chemical characteristics of the device, aroma from the device may spread throughout an area such as a room in a house or an interior of a car. Conventional aromatic devices come in various styles including surface-mountable, standing, hanging, and heat activated.

Some conventional surface-mountable aromatic devices are mountable on surfaces such as walls. One brand of surface-mountable device is a Stick Ups air freshener, which includes a scented pellet, a rigid vented body surrounding the pellet allowing scent to pass through the vents and away from the body, and double-sided tape attached to a back of the body for mounting the device to a wall. Stick Ups is a federally registered trademark of Airwick Industries, Inc., of Carlstadt, N.J. Standing air fresheners are designed to stand on a surface such as a bathroom counter. Exemplary standing containers include containers of potpourri, devices including a plastic vented base encasing a scented material, and candles that emit aroma.

Some aromatic devices include a loop for hanging them from structures such as a rear-view mirror of an automobile or a hook in a gym locker. Heat-activated aromatic devices include materials that emit an aroma when heated, such as by flame or electric current. Exemplary heat-activated aromatic devices include candles, incense, and oils. One brand of electrically heated aromatic device is a Plug-Ins air freshener, which includes a vented casing including a cartridge holding fragranced gel and prongs connected to the cartridge and extending from the casing for plugging the device into a standard electrical outlet. Plug-Ins is a federally registered trademark of S.C. Johnson & Sons, Inc., of Racine, Wis. The electricity passing between the prongs and contacting the gel heats the gel, which emits an aroma when heated.

Conventional aromatic devices have various drawbacks. Most aromatic devices cannot be mounted on surfaces and therefore have limited utility. These devices that cannot be mounted on surfaces are inconvenient to use, especially in areas such as an office or a kitchen where desk and counter space are limited. Conventional aromatic devices including a string for hanging the device from a car mirror or locker hook are generally limited to use at locations where the device can be hung. Further, conventional devices, surface mountable or not, are generally relatively large. Thus, whether mounted on a surface or positioned on a countertop, conventional aromatic devices can be awkward and take up too much space. Relatively small surface-mountable aromatic devices would be useful and convenient.

Most surface-mountable aromatic devices are designed to be mounted only once. For example, devices mountable by adhesive such as glue or double-sided tape are generally designed to be mounted once. Removing one of these conventional devices from a surface on which it is mounted often damages the device and/or the surface, such as by leaving unsightly residue or peeling paint or wall paper away from the surface. Further, removing a nail or tack used to attach a device to a surface leaves marks on the surface. Aromatic devices are sought that are easily mountable, removable, and re-mountable for locating and relocating the device as desired.

One drawback of aromatic devices requiring heat to emit aroma is that a user of the device must provide heat, such as by electric circuit or flame. This energy requirement limits use of the device in many ways. For example, aromatic devices needing to be plugged into an electrical outlet for operation can only be positioned for use at a location having an outlet. As another example, candles can only be burned in certain safe areas, such as areas having sufficient ventilation and being free of nearby flammable materials. Further, the energy required to operate these devices, such as from electricity, lighters, and matches, increases the effective cost of the device. As described herein, the aromatic suction-mountable devices of the present invention do not have the aforementioned drawbacks.

There are many suction-mountable devices, such as conventional suction cups, but they are not aromatic. Conventional suction-mountable devices are very convenient for purposes including decoration because they can be formed in a variety of shapes, positioned on most any generally smooth surface, and easily relocated as desired. Further, many suction-mountable devices include a hook for holding items such as keys. Also, art and other messages can be formed on the body of suction-mountable devices. Despite all of these benefits, conventional suction-mountable devices do not freshen the air like the conventional aromatic devices described above.

### BRIEF SUMMARY OF THE INVENTION

In one aspect, the present invention relates to a deformable aromatic device mountable by suction on a generally smooth mounting surface including a body including a flexible base formed from a first material. The flexible base has an outer surface, an inner surface including a concave portion opposite the outer surface, and a periphery extending around the body, surrounding the outer surface and the inner surface of the body. The body further includes a message formed on the body at a position selected from a group of positions consisting of an outer position on the outer surface, an inner position on the inner surface, and an intermediate position between the outer surface and the inner surface. The device further includes a second material selected to emit a desired aroma connected to the base. The body is deformable from an undeformed shape to an actuated shape to mount the body on the mounting surface by positioning the inner surface of the body adjacent the mounting surface when the body is in the undeformed shape thereby forming a cavity between the concave portion and the mounting surface and deforming the body to its actuated shape thereby sealing the body against the mounting surface and forcing fluid out of the cavity to create suction between the body and the mounting surface thereby securing the body to the mounting surface.

In another aspect, the present invention relates to a deformable aromatic device mountable by suction on a generally smooth mounting surface including a body including a flexible base formed from a first material. The flexible base has an outer surface, an inner surface including a concave portion opposite the outer surface, and a periphery extending around the body, surrounding the outer surface and the inner surface of the body. The device further includes a protrusion extending from the outer surface of the body and the protrusion includes a second material emitting a desired aroma. The body is deformable from an undeformed shape to an actuated shape to mount the body on the mounting surface by positioning the inner surface of the body adjacent the mounting surface when the body is in the undeformed shape thereby forming a cavity between the concave portion and the mounting surface and deforming the body to its actuated shape thereby sealing the body against the mounting surface and forcing fluid out of the cavity to create suction between the body and the mounting surface thereby securing the body to the mounting surface.

In yet another aspect, the present invention relates to a deformable aromatic device mountable by suction on a generally smooth mounting surface including a body including a flexible base formed from a first material. The flexible base has an outer surface, an inner surface including a concave portion opposite the outer surface, and a periphery extending around the body, surrounding the outer surface and the inner surface of the body. The device further includes a second material selected to emit a desired aroma connected to the base wherein the aroma corresponds to a shape of the periphery of the body. The body is deformable from an undeformed shape to an actuated shape to mount the body on the mounting surface by positioning the inner surface of the body adjacent the mounting surface when the body is in the undeformed shape thereby forming a cavity between the concave portion and the mounting surface and deforming the body to its actuated shape thereby sealing the body against the mounting surface and forcing fluid out of the cavity to create suction between the body and the mounting surface thereby securing the body to the mounting surface.

In still another aspect, the present invention relates to a deformable aromatic device mountable by suction on a generally smooth mounting surface including a body including a flexible base formed from a first material. The flexible base has an outer surface, an inner surface including a concave portion opposite the outer surface, and a periphery extending around the body, surrounding the outer surface and the inner surface of the body. The device further includes a protrusion extending from the outer surface of the body including an exterior surface and a periphery extending around the protrusion and surrounding the exterior surface. The device also includes a second material selected to emit a desired aroma connected to the base wherein the aroma corresponds to a shape of the periphery of the protrusion. The body is deformable from an undeformed shape to an actuated shape to mount the body on the mounting surface by positioning the inner surface of the body adjacent the mounting surface when the body is in the undeformed shape thereby forming a cavity between the concave portion and the mounting surface and deforming the body to its actuated shape thereby sealing the body against the mounting surface and forcing fluid out of the cavity to create suction between the body and the mounting surface thereby securing the body to the mounting surface.

In yet still another aspect, the present invention relates to a deformable aromatic device mountable by suction on a generally smooth mounting surface including a body including a flexible base formed from a first material and having an outer surface, an inner surface including a concave portion opposite the outer surface, and a non-circular periphery extending around the body, surrounding the outer surface and the inner surface of the body. The device further includes a second material selected to emit a desired aroma connected to the base wherein the aroma corresponds to a shape of the periphery of the body. The body is deformable from an undeformed shape to an actuated shape to mount the body on the mounting surface by positioning the inner surface of the body adjacent the mounting surface when the body is in the undeformed shape thereby forming a cavity between the concave portion and the mounting surface and deforming the body to its actuated shape thereby sealing the body against the mounting surface and forcing fluid out of the cavity to create suction between the body and the mounting surface thereby securing the body to the mounting surface.

In a further aspect, the present invention relates to a deformable aromatic device mountable by suction on a generally smooth mounting surface including a body including a flexible base formed from a first material. The flexible base has an outer surface, an inner surface including a concave portion opposite the outer surface, and a periphery extending around the body, surrounding the outer surface and the inner surface of the body. The device further includes a second material selected to emit a desired aroma connected to the base wherein the aroma corresponds to a shape of the periphery of the body. The device also includes a fastener attached to the outer surface of the body for attaching an item to the device. The body is deformable from an undeformed shape to an actuated shape to mount the body on the mounting surface by positioning the inner surface of the body adjacent the mounting surface when the body is in the undeformed shape thereby forming a cavity between the concave portion and the mounting surface and deforming the body to its actuated shape thereby sealing the body against the mounting surface and forcing fluid out of the cavity to create suction between the body and the mounting surface thereby securing the body to the mounting surface.

In another aspect, the present invention relates to a method of using an aromatic device including a deformable body having an outer surface, an inner surface having a concave portion opposite the outer surface, a periphery extending around the body and surrounding the inner surface and the outer surface, and a material emitting a desired aroma. The method includes positioning the inner surface of the body adjacent a mounting surface in an area occupied by people or used by people to store things so the aroma emitted from the body may be sensed by the people when they are in or near the area. The inner surface of the body is positioned adjacent the mounting surface while the body is in an undeformed shape thereby forming a cavity between the body and the mounting surface. The method further includes moving the body to an actuated shape thereby sealing the body against the mounting surface and forcing fluid out of the cavity to create suction between the body and the mounting surface thereby securing the body to the mounting surface in the area.

Other aspects of the present invention will be in part apparent and in part pointed out hereinafter.

### BRIEF DESCRIPTION OF THE DRAWINGS

FIG. 1 is a plan of an aromatic suction-mountable display device according to a first embodiment of the present invention.

FIG. 2 is a cross section of the aromatic suction-mountable display device taken along line 2-2 of FIG. 1 shown in a default shape adjacent a mounting surface.

FIG. 3 is a cross section of the aromatic suction-mountable display device shown in an actuated shape mounted on the mounting surface.

FIG. 4 is a cross section of the aromatic suction-mountable display device shown mounted on the mounting surface and holding an object by way of a fastener.

FIG. 5 is a cross section of the aromatic suction-mountable display device shown mounted on the mounting surface and holding an object by way of another fastener.

FIG. 6 is a plan of an aromatic suction-mountable display device according to a second embodiment of the present invention.

FIG. 7 is a plan of an aromatic suction-mountable display device according to a third embodiment of the present invention.

FIG. 8 is a plan of an aromatic suction-mountable display device according to a fourth embodiment of the present invention.

FIG. 9 is a perspective of an aromatic suction-mountable display device according to a fifth embodiment of the present invention.

FIG. 10 is a perspective of an aromatic suction-mountable display device according to a sixth embodiment of the present invention.

FIG. 11 is a perspective of an aromatic suction-mountable display device according to a seventh embodiment of the present invention.

FIG. 12 is a perspective of an aromatic suction-mountable display device according to an eighth embodiment of the present invention.

Corresponding reference characters indicate corresponding parts throughout the several views of the drawings.

### DETAILED DESCRIPTION OF THE INVENTION

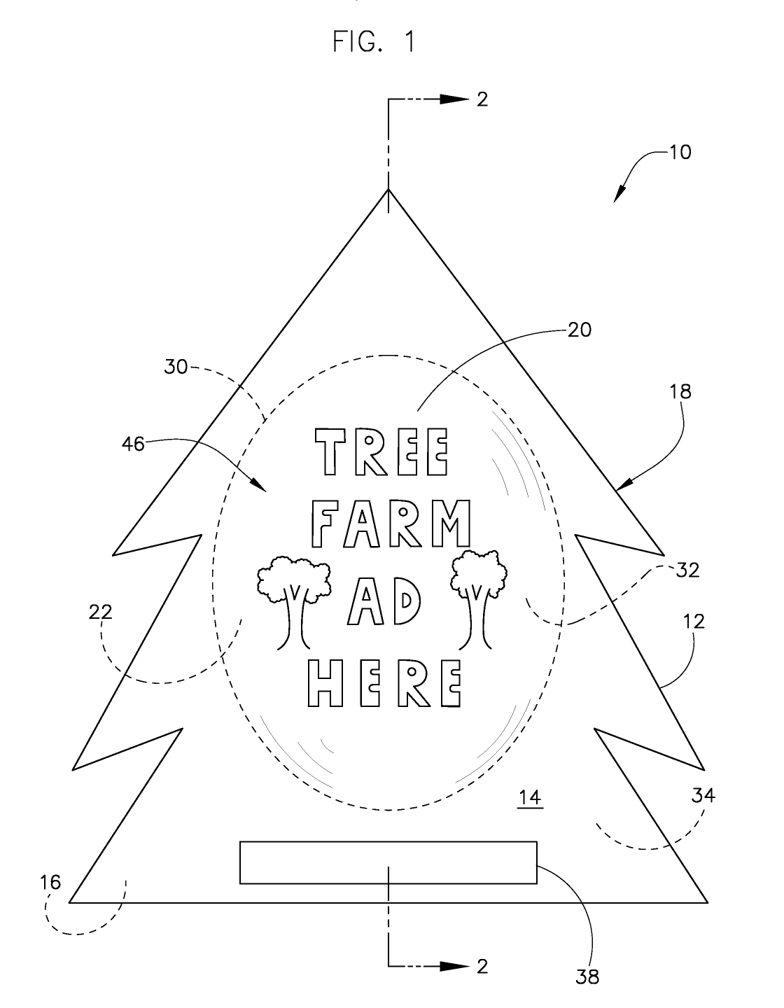

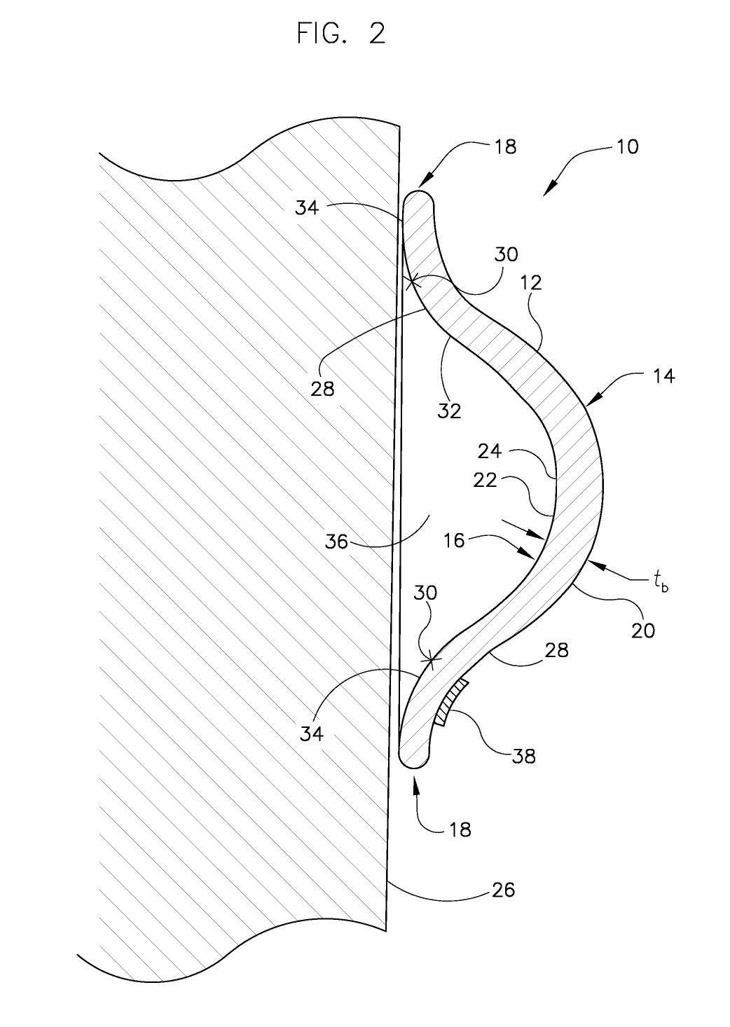

Referring to the figures, and more particularly to FIG. 1, a portable aromatic device according to a first embodiment of the present invention is designated in its entirety by reference number 10. The aromatic device 10 includes a flexible or deformable body 12 having an outer surface 14, an inner surface 16 opposite the outer surface, and a periphery 18 extending around the body and surrounding the inner surface and the outer surface. Although the flexible body 12 may include other base materials without departing from the scope of the present invention, in one embodiment the body includes a resinous thermoplastic such as polyvinyl chloride as a flexible base material. Other exemplary base materials for the body 12 are described below. The body 12 also includes material (not shown in detail) selected to emit a desired scent or aroma. The aromatic material of the body 12 is described in further detail below. As shown in FIG. 2, the outer surface 14 may include a convex portion 20 and the inner surface 16 includes a concave portion 22 opposite the outer surface. The concave portion 22 of the inner surface 16 has a center 24. For embodiments in which the outer surface 14 includes the convex portion 20, the concave portion 22 of the inner surface 16 may be opposite the convex portion.

The periphery 18 of the body 12 may be generally circular or non-circular. In one embodiment, the periphery 18 is non-circular and non-oval. The periphery 18 may be symmetric, such as circular, square, or oval, or non-symmetric. For example, the periphery 18 of the body 12 shown in FIG. 1 is non-circular and symmetric and, specifically, shaped as a pine tree. The shape of the body may represent an article or thing. The terms “article” and “thing” are used in broad senses and exemplary articles and things include but are not limited to a person, an animal, common geometric shapes such as a triangle and a hexagon, a vehicle such as a car or airplane, a building, and everyday articles such as a clock or a tennis racket.

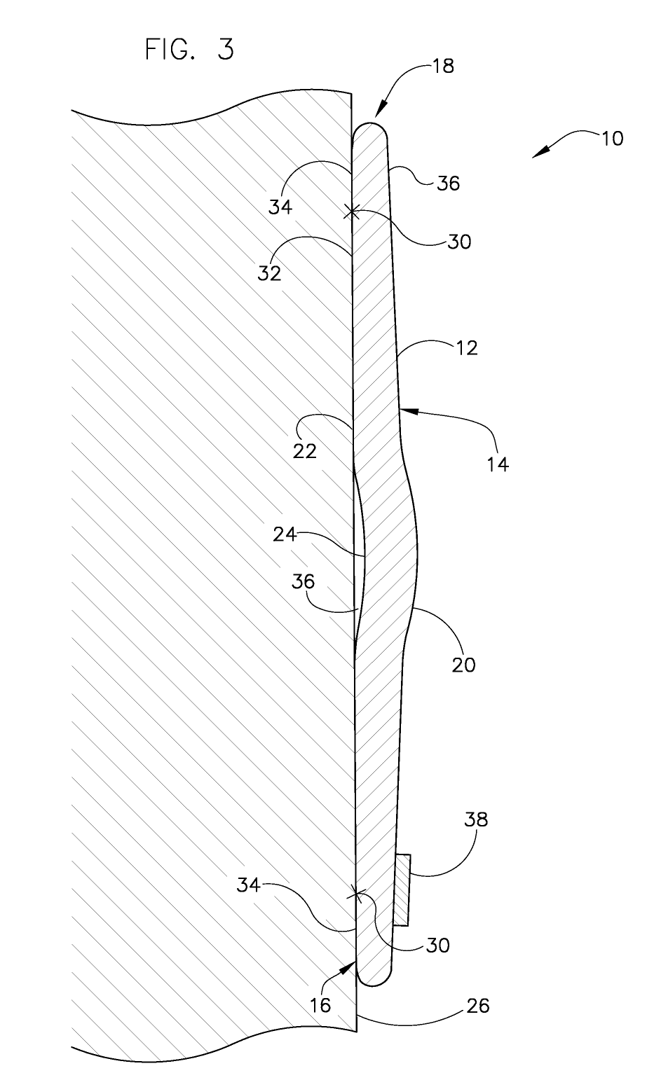

Circular bodies 12 are common because reflex forces developed within them are naturally distributed in a uniform circular pattern promoting a required balance between reflex forces and suction forces created between the body and a mounting surface 26 for mounting the device 10 on the mounting surface. As the concave flexible body 12 is deformed toward an actuated shape (shown in FIG. 3), reflex or spring forces are created in the body between the convex and concave portions 20, 22 that act to return the body to its default shape. As will be appreciated by those skilled in the art, the flexible body 12 must be sized and shaped so forces created by the suction between the body and the mounting surface 26 balance with or are greater than the sum of the reflex forces created in the body and any loading on the body to create and maintain the connection between the body and the mounting surface.

The flexible body 12 has a thickness t_{b} measured between the outer surface 14 and the inner surface 16 of the body. The thickness t_{b} may be generally constant or vary throughout the flexible body 12. One way to reduce the effect of non-circular reflex forces in the body 12 is to taper (e.g., steadily decrease) the thickness t_{b} of the body between the center 24 of the concave portion 22 of the inner surface 16 and the periphery 18 of the body. For example, the thickness t_{b} of the body 12 may vary from a maximum thickness of between about 0.10 inch and about 0.25 inch adjacent the center 24 of the concave portion 22 of the inner surface 16 and a minimum thickness of between about 0.045 inch and about 0.125 inch adjacent the periphery 18. The reflex forces developed in tapered bodies 12 during deformation of them are more uniform because there is less material nearer the periphery 18 where the body is non-uniformly shaped. The body 12 near the periphery 18 contributes less to the reflex forces of the body in proportion to the decreased thickness, allowing the reflex forces distributed more uniformly around a center of the body to predominate the sum of the reflex forces. By developing more uniform reflex forces in the body 12, devices 10 having a tapered thickness t_{b} can securely mount on mounting surfaces 26.

It is contemplated that reflex forces may also be reduced by varying the material or material properties of the flexible body 12 between the center 24 and the periphery 18. For example, the material of the body 12 may be less resilient nearer the periphery to reduce reflex forces created by the body adjacent the periphery.

Another way to reduce the effect of non-circular reflex forces is by forming the body 12 so the inner surface 16 includes a bend 28 (shown in FIG. 2) adjacent a boundary 30 of a suction portion 32 of the inner surface. The bend 28 may be positioned between the boundary 30 of the suction portion 32 and the periphery 18 of the body 12. The suction portion 32 consists of the surface of the body 12 that interacts with the mounting surface 26 to create the suction mounting the device 10 on the mounting surface. The suction portion 32 includes the concave portion 22 of the inner surface 16 and the boundary 30 extends around and defines the suction portion. The body 12 further includes an additional portion 34 between the suction portion 32 and the periphery 18. The additional portion 34 of the body 12 is not needed to create the suction used to mount the device 10 on the mounting surface 26. The body 12 may include the additional portion 34 even if the inner surface 16 of the body does not include the bend 28. Although the bend 28 may change a direction of the inner surface 16 by other amounts when the flexible body 12 is undeformed, in one embodiment the bend changes the direction of the inner surface by between about 5° and about 20° when the flexible body is undeformed. The body 12 may have any combination of a tapered body thickness t_{b}, varying body material, varying material properties, and the bend 28 adjacent the boundary 30 of the suction portion 32 of the inner surface 16. For example, the body 12 may have a tapered thickness t_{b}, generally homogenous body material and material properties, and no bend 28 in the inner surface 16 of the body.

The flexible body 12 is deformable between an undeformed or default shape, shown in FIG. 2, and an actuated shape, shown in FIG. 3, to mount the display device 10 by suction or vacuum on the mounting surface 26. The display device 10 may be secured to mounting surfaces 26 having various attitudes. For example, the display device 10 may be mounted on an upward facing or downward facing horizontal mounting surface 26. The display device 10 may also be mounted on a vertical surface 26 or on a generally upward or downward facing surface angled between horizontal and vertical. A mounting strength of the display device 10 depends in part on the size, shape, and material of the display device and characteristics of the mounting surface 26, such as smoothness.

The mounting surface 26 must include a surface area that is at least as large as an area spanned by the suction portion 32 of the inner surface 16 of the body 12 when the flexible body is in its actuated shape. Exemplary mounting surfaces 26 including mounting surfaces in an area occupied by people or used by people to store things so the aroma emitted from the body may be sensed by the people when they are in or near the area. For example, the device 10 may be mounted on a generally smooth interior surface of an automobile or other vehicle (e.g., boat and aircraft), a surface (e.g., interior) of a locker, a wall, a window, a mirror, a component of a computer, or a desk. The device 10 may be mounted to a surface 26 in a variety of environments including outdoors, in a bathroom, kitchen, basement or other part of a house or building such as a store, restaurant, or office.

To mount the display device 10 on the mounting surface 26, the inner surface 16 of the flexible body 12 is positioned adjacent the mounting surface when the body is in its default shape, as shown in FIG. 2. By positioning the flexible body 12 in this way, a cavity 36 is formed between the suction portion 32 of the body and the mounting surface 26. After the flexible body 12 is positioned adjacent the mounting surface 26 as described, the flexible body is moved to its actuated shape (i.e., causing the inner surface 16 to conform to the mounting surface), as shown in FIG. 3, thereby sealing the body against the mounting surface and forcing fluid such as air out of the cavity 36. The suction created within the sealed cavity 36 secures the body 12 to the mounting surface 26. As the flexible body 12 is deformed toward the actuated shape, reflex or spring forces are created in the body between the convex and concave portions 20, 22 that act to return the body to its default shape thereby creating the suction between the body and the mounting surface 26. As will be appreciated by those skilled in the art, the flexible body 12 must be sized and shaped so forces created by the suction are balanced with or are greater than the sum of the reflex forces created in the body and any loading on the body to create and maintain the connection between the body and mounting surface 26.

To demount the flexible body 12 from the mounting surface 26, for example to relocate the display device 10 on another smooth mounting surface, the body is moved out of its actuated shape thereby breaking the seal and suction. To break the seal and move the body 12 out of its actuated shape, the user may move or pull on a portion of the periphery 18 of the body. Demounting the body 12 from the mounting surface 26 is both clean and simple. In contrast, removing a conventional aromatic device mounted on a surface by adhesive from that surface is more involved and often results in damage to the surface and/or the device, as described in the Background of the Invention section.

As described above, the body 12 includes a flexible base material. As a base material, the body 12 may include a polymer such as a rubber or an elastomeric material. The base material for the body 12 may include thermoplastics or other plastics. Particular examples of base materials for the body 12 are silicone, polyurethane, polyvinyl chloride or another polyvinyl or vinyl, neoprene, formed by polymerization of chloroprene, and nitrile or Buna-N, which is a copolymer of butadiene and acrylonitrile. The base material of the body 12 may be a single material or a combination of materials. The particular base material used depends on desired performance characteristics such as resilience, texture, and appearance, including color. The body 12 must be somewhat resilient and elastic so the body acts to return to the default shape when it is moved to the actuated shape thereby pressing against the mounting surface 26 and creating the suction used to mount the device on the mounting surface as described above. However, if the body 12 is too resilient, it will be too difficult to move the body to the undeformed shape and/or reflex forces greater than the suction will be created between the body and the mounting surface 26 thereby breaking the seal and suction securing the device 10 to the mounting surface.

As also described above, the body 12 includes a material (not shown in detail) selected to emit a desired aroma or scent. The scented material of the body 12 may include a single component, such as a single fragrant oil, or may include a combination of components. Various scents may be used without departing from the scope of the present invention. For example, the scented material may emit an aroma selected from a group of aromas including floral scents, fruit scents, spices, herbs, other natural scents, and combinations of these scents. Exemplary floral scents include jasmine, lilac, lily, rose, ylang-ylang, magnolia, orchid, cherry blossom, and honeysuckle. Exemplary fruit scents include citrus, coconut, orange, banana, lemon, watermelon, cherry, strawberry or other berries, mango, watermelon, pineapple, kiwi, and grapefruit. Exemplary spices include cinnamon, coffee, and nutmeg. Exemplary herbs include rosemary, lemongrass, bergamot, chamomile, and mints such as sage, basil, oregano, thyme, peppermint, and lavender. Other natural scents include sandalwood, cucumber, patchouli, vanilla, eucalyptus, and pine. Further, the scented material may also emit other common aromas such as bubble gum, cotton candy, baby powder, musks, and chocolate. In addition, the aroma may resemble a popular perfume or cologne of a newly created perfume or cologne.

Although the scented material may have other forms, in one embodiment the scented material is in the form of a gel, a powder, or a liquid such as an oil. In one embodiment, the scented material of the body 12 is a liquid concentrate. The scented material may be added to the base material of the body 12 at various stages of development of the device 10. In one embodiment, the scented material is coated onto the outer surface 14 and/or the inner surface 16 of the body 12. Although the scented material may be coated onto the outer surface 14 and/or the inner surface 16 of the body 12 in other ways without departing from the scope of the present invention, in one embodiment the scented material is coated onto the outer surface and/or the inner surface of the body by dipping the body into a bath of the scented material, spraying the body with the scented material, or painting the body with the scented material such as by brush or other applicator. It is contemplated that the material emitting an aroma may also be part of or formed on a thin item or film such as a label or sticker attached to the outer surface 14 of the body 12.

In one embodiment, the scented material is impregnated into the body 12 during manufacture of the device 10. The scented material may be added to the base material before the base material is molded and/or cured to form the resulting shaped body 12. For example, in one embodiment the scented material in the form of an oil is added to and mixed with a liquid or gel silicone base material before molding and curing the base material to form the shaped body 12.

The molding and curing steps of forming flexible vacuum-mountable devices are well known and include positioning the base material and any processing agents into a mold, such as by injection, and setting or curing that material to form the resulting body 12. As will be apparent to those skilled in the art, an inhibitor or an accelerator may be added to the mixture forming the body 12 to control the rate and manner at which the body is molded and cures. Also, the base material of the body 12 may be vulcanized during formation by adding select chemical agents to add useful properties to the body or enhance such properties in the body including, for example, elasticity, strength, and stability. Vulcanizing materials include activators to initiate vulcanization, fillers, such as carbon, to reinforce the structure thereby enhancing chemical and mechanical properties of the body 12, and vulcanization agents to cross-link polymers. Other agents that may be added to the body material to affect the formation process and characteristics of the resulting body 12 include plasticizers, which soften the material and/or improve processing, processing aids, which may ease handling during mixing, extrusion, calendaring, or molding, mold release agents, which may be coated onto the mold such as by spraying and make it easier to remove the bodies from the mold they are molded in, and age-resistors, which retard aging of the body. In addition, as described in further detail below, pigments may be added to the base material to color the body 12 as desired.

The base material and the scented material of the body 12 and the manner in which they are combined and processed affect characteristics of the device 10 such as mounting and aroma emitting characteristics. In one embodiment, the base body material is selected, the scented material is selected and added to the body 12, and the body is processed so the device 10 emits a relatively strong aroma. In another embodiment, the base material is selected, the scented material is selected and added to the body, and the body is processed so the device emits aroma for a relatively long period of time (e.g., over one year). Although emission of aroma from the body 12 may be controlled in other ways without departing from the scope of the present invention, in one embodiment the emission of aroma from the body is controlled by controlling a rate of evaporation of the scented material. The rate of evaporation or diffusion of the scented material depends on the substance of the scented material because various substances evaporate in different ways. For example, one scented material may evaporate rapidly when exposed to air, thus providing a strong aroma for a relatively shorter period of time, and another scented material may evaporate more slowly, providing an aroma for a longer period of time. Also, it is believed that the rate of evaporation may be controlled by controlling the presence and configuration of voids formed in the base material of the body 12 and channels connecting the voids to the each other and to the surface. By experimentation that is within the ability of those skilled in the art, a designer of the device 10 can select particular base materials, scented materials, and processing agents and methods to make an aromatic suction-mountable device having desired characteristics. For example, the designer may experiment with materials and processes to create a device 10 with desired performance characteristics such as ability to adhere to a variety of surfaces and emit a strong aroma for a relatively long period of time.

As mentioned above, the body 12 may be colored or tinted as desired. One manner of coloring the body is by adding pigment to the base material of the body during the formation process. Another manner of providing a body having a desired color is to select a base material inherently having certain color characteristics. For example, some flexible base materials have a default color, such as blue, black, red, green, white, orange, tan, grey, brown, purple, or variations of these colors, such as a translucent green or a brownish red.

As described above, the additional portion 34 of the inner surface 16 between the boundary 30 of the suction portion 32 and the periphery 18 is not needed to create the suction used to mount the device 10 on the mounting surface 26. The additional portion 34 and the periphery 18 surrounding the additional portion may have various sizes and shapes without departing from the scope of the present invention, as long as the suction between the body 12 and the mounting surface 26 can be created and maintained as described above. Exemplary periphery 18 shapes include those corresponding to articles of clothing such as a shirt or shoe, structures such as a building or bridge, a vehicle such as a car, boat, or airplane, and articles of nature such as a fruit, flower, tree, person, or animal. Other exemplary periphery 18 shapes are described below with respect to additional embodiments of the present invention.

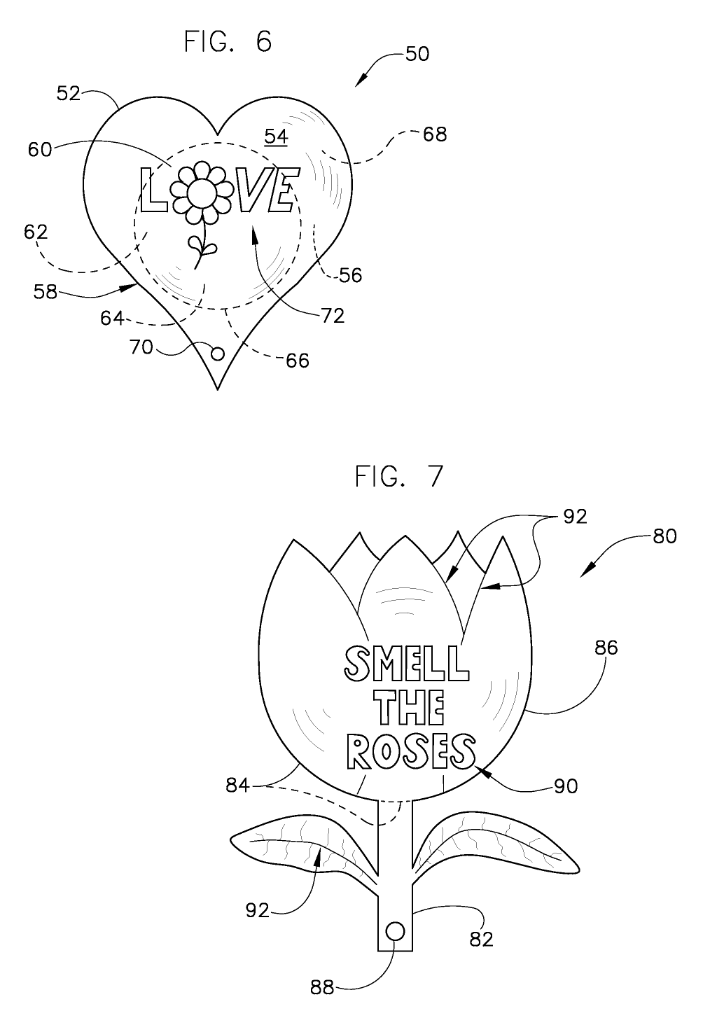

The color or tint of the body 12, a shape of the periphery 18 of the body, and/or the aroma emitted by the body may relate to each other in various combinations. For example, the body 12 having a periphery 18 shaped as a pine tree shown in FIG. 1 may be colored or tinted green. As another example, a body 12 shaped as an apple (not shown) or a heart (such as the body 52 shown in FIG. 6) may be colored or red corresponding to a common color of apples and heart-shaped objects. As yet another example (not shown), a body 12 shaped as a banana may be colored yellow and include a material emitting a banana aroma. As still another example (not shown), a body 12 shaped as a bumble bee may be colored with alternating yellow and black stripes and include a material emitting a honey aroma. In this example, the coloring of the body 12, the aroma emitted by the body, and the shape of the periphery 18 all relate to each other. The honey aroma and the bumble bee shape of the body 12 relate to each other because bees make honey, the colored stripes relate to the bee body shape because they both contribute to the image of the bee, and the honey aroma relates to the colored stripes because the stripes are forming a pattern reminiscent of the body of bees, which make honey.

The shape of the periphery 18 of the body 12 may be designed to communicate a message or theme. As an example, in one embodiment (not shown) the periphery 18 is shaped as an arrow and may be used to, for example, indicate a particular direction or point to an item. For instance, an arrow-shaped device including material that glows in the dark can be secured to a wall adjacent and pointing to a door in a darkened room such as a movie theater or an x-ray room. In this example, the arrow-shaped device would indicate a location of the door or doorknob. In another embodiment (not shown) of a device 10 having a periphery 18 forming a message or theme, the periphery is shaped as words, numbers, a logo, or other symbol or character. For example, the periphery 18 may be shaped to form the letters “LOVE”. As another example, the periphery 18 may form numbers, such as a two digit number representing, for instance, a jersey number of a famous professional athlete of a franchise or a channel number of a television channel using the device 10 for marketing.

In one embodiment, the periphery 18 of the body 12 has a shape relating to an entity using the device, such as a store or restaurant. For example, the periphery 18 of the body 12 may be shaped as a piece of fruit and used at a fruit market and/or as an advertisement for the market. Fruit-shaped peripheries may communicate, among other things, that fruit is sold in the area where the devices are mounted. As another example, the periphery 18 may be shaped as a fish to indicate that produce and seafood are sold in the area where the device is mounted. The particular periphery 18 shapes relating to an entity described herein are only exemplary and the device 10 may be designed to include peripheries having shapes relating to various other entities in various ways.

The shape of the periphery 18 may also communicate a theme of a company or a person's trade. As an example (not shown), the periphery 18 may be shaped as a dolphin for communicating, among other things, that the user of the device works with such animals. A dolphin-shaped periphery may also communicate, among other things, that the person using the device is fond of dolphins. As another example, the periphery 18 may be shaped as a house and used by a real estate agent or home builder to communicate their trade and/or for advertising purposes. The particular messages that the periphery 18 shapes may communicate described herein are only exemplary and the message and themes that the device may be designed to communicate are not limited to these examples.

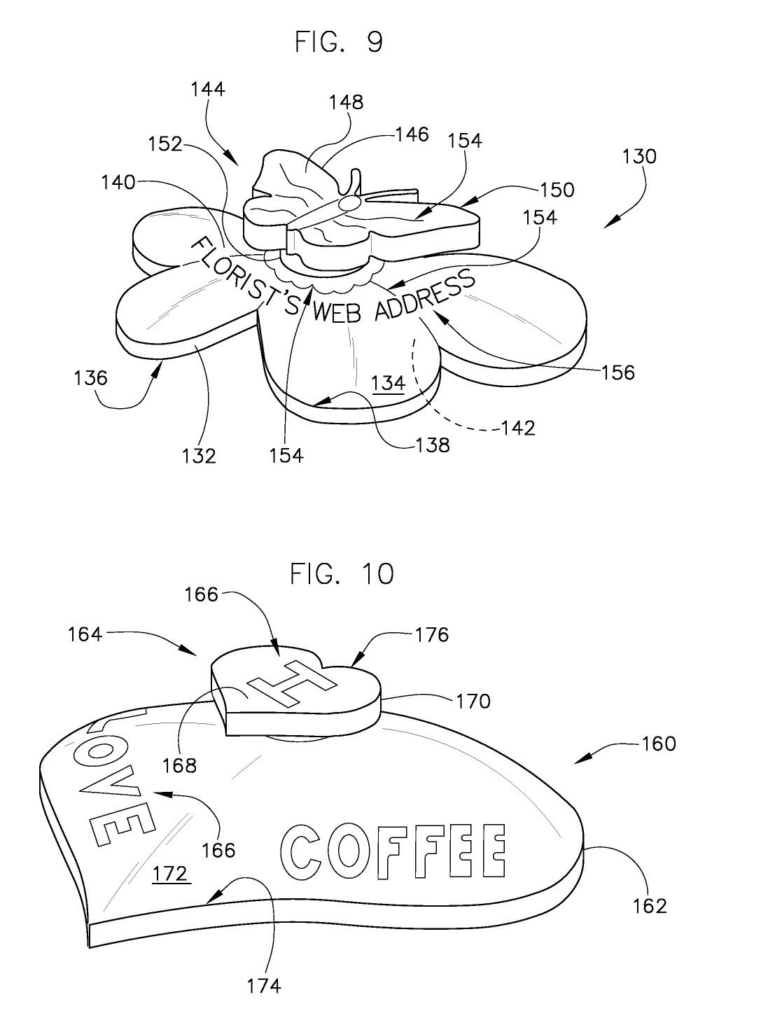

As described above, the aroma emitted from the body 12 may correspond to the shape of the periphery 18 of the body. Specifically, when the shape of the periphery 18 of the body 12 communicates a message or theme, that shape may correspond to the aroma emitted by the body 12. In one embodiment, the shape of the periphery 18 of the body 12 is shaped as an item of nature and the body includes a material emitting an aroma resembling the item. For example, the periphery 18 may be shaped as a fruit and the body 12 may include material emitting an aroma of that fruit. As another example, the periphery 18 may be shaped as a flower (such as the peripheries 86, 138 in FIGS. 7 and 9) and the body 12 may emit a corresponding floral aroma. As yet another example, in one embodiment (not shown) the body 12 is shaped as a piece of candy or bubble gum and the body emits a candy scent, such as cinnamon, or a bubble gum scent, respectively. As still another example, the body 12 may have a periphery 18 shaped as a coffee cup and include material emitting a coffee bean aroma. The device 10 of this example may also include a message such as “COFFEE HOUSE” printed on the outer surface 14 and be used, for example, as a marketing device for a coffee company or café. As shown by this example, the body 12 may include a message formed on or in the body corresponding to the aroma emitted by the body. This concept is described in further detail below.

In one embodiment the periphery 18 forms a shape related to an entity such as a store or restaurant. As shown in FIG. 1, the periphery 18 of the body 12 is shaped as a pine tree. The device 10 according to this embodiment may be used as, for example, a marketing tool for a nursery or tree farm. The aroma emitted by the body 12 may be a pine aroma corresponding to the message or theme of the nursery, which includes the sale of trees and is symbolized by the tree shape of the periphery 18. As another example, in one embodiment (not shown) the periphery 18 of the body 12 is shaped as a pot or a cooking hat communicating a food-related theme or message of a restaurant or cook and includes material emitting a food-related aroma such as a scent of a selected spice or herb corresponding to the food-related theme or message.

As yet another example, in one embodiment (not shown) the body 12 has a periphery 18 shaped as a coffee cup and includes material emitting a coffee bean aroma corresponding to the coffee-cup shape of the body, which may relate to the business of a coffee making or coffee vending company. Such a device 10 may include a message such as “COFFEE HOUSE” printed on the outer surface 14 of the body and be used, for example, as a marketing device for a coffee company or cafe. As still another example of the aroma emitted by the body corresponding to the shape of the body 12, which may communicate a theme or message related to an entity, in one embodiment (not shown) the body is shaped as a bucket of popcorn and includes the name of a movie theater or popcorn-making company formed on the outer surface 14 and a material emitting an aroma of popcorn (e.g., buttered popcorn).

Devices 10 according to these and other embodiments may include markings or accents (such as the markings 92, 154, 190, 210 shown in FIGS. 7, 9, 11, and 12) formed on or in the body 12. The markings may be formed on the outer surface 14, on the inner surface 16, and/or between the outer and the inner surfaces of the body 12. The markings and accents may be printed on a label adhered to the outer surface 14 and the label may be fully or partially opaque, translucent, and/or transparent as desired. Further, the markings or accents may be formed using material of the flexible body 12 by, for example, molding, melting, etching, engraving, doping, or impressing. The markings may correspond to the shape of the periphery 18 of the body 12 and may be formed in any desired color. For example, in one embodiment (not shown) the periphery 18 of the body 12 is shaped as a head of a person and includes colors and markings printed on the outer surface 14 of the body forming details (e.g., eyes, nose, and mouth) of the person's face.

As another example of the markings or accents relating to a shape of the body 12, in the embodiment of the device 10 having a periphery 18 shaped as a number of an athlete described above, the body may include details further defining the number formed by the periphery by interior lines of the numbers between the periphery. As another example, a body 12 shaped to form the word “LOVE” may be colored red, include markings in the form of interior lines printed on the outer surface 14 of the body further defining the letters, and include accents in the form of raised material giving the heart a rounded surface and thereby more of a three-dimensional appearance. As other examples, the body 12 having periphery 18 shapes described above, such as a football helmet, and a dolphin, may also include markings and/or accenting related to the shape of the periphery. Thus, a football-shaped body (not shown) may be colored brown and include an elevated portion forming dimples and an elevated portion colored white forming laces of the football. The particular periphery 18 shapes communicating messages described herein are only exemplary and the device 10 may be designed to include peripheries communicating many other things in various ways.

In addition to the shape of the periphery 18, the aroma emitted by the body 12 may correspond to the markings or accents formed on the body. The accents may include textured, raised, and lowered portions of the base body material or other material attached to the body 12. For example, an ice cream cone-shaped body 12 (not shown) may be colored brown at a cone portion of the body and cream or white at an ice cream portion of the body and may include accents and/or markings (such as the markings 92, 154, 190, 210 shown in FIGS. 7, 9, 11, and 12) forming details of the ice cream cone. The markings in this embodiment may include lines between adjacent scoops of ice cream at the ice cream portion of the body 12. The accents in this embodiment may include raised and/or lowered portions of body material creating a three-dimensional effect for the ice cream. Further in this embodiment, the body may include a material emitting a vanilla scent corresponding to the ice cream cone shape of the periphery 18 and corresponding to the color, markings, and accents forming the ice cream cone image.

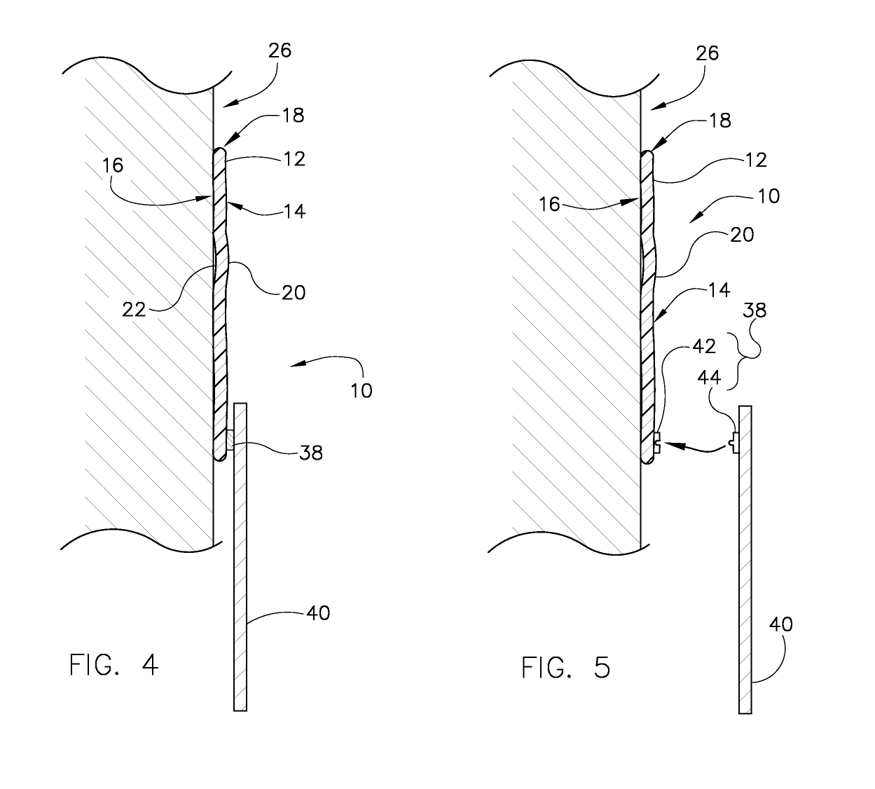

As shown in FIGS. 1-5, the device 10 may include a connector or fastener 38 used for attaching an item to the body 12. The connector 38 may be formed on the outer surface 14 of the body 12 opposite the additional portion 34 of the inner surface 16 or opposite the suction portion 32 of the inner surface. FIG. 4 shows the device 10 holding an item or object 40 such as a calendar or a dry-erase board. The device 10 may include various types of connectors 38 without departing from the scope of the present invention. In one embodiment, the connector 38 is selected from a group of fasteners including of snap, hook-and-loop or hook-and-pile, adhesive, and magnet. FIG. 5 shows the device 10 including a snap or a magnet connector 38 including a receiving part 42 attached to the body 12 and a mating button part 44 attached to the item 40. Other exemplary releasable connectors 38 include an adhesive and a latch-type connector.

The embodiments of the device 10 including the fastener 38 may be used to hold a variety of items 40 without departing from the scope of the invention. In addition to the calendar and dry-erase board, exemplary items 40 include a photo or photo display frame, art, a pad of paper, an agenda, and a schedule (e.g., a schedule of a professional athletic team). When the device 10 is used to hold items that can be written on, such as a calendar, a pad of paper, or dry-erase board, it is contemplated that the device may also hold a writing instrument (not shown), such as a pencil, pen, or dry-erase marker. The writing instrument may be connected to the device 10, such as by way of a string or cord and a hole (similar to the hole 70 shown in FIG. 6) through the body 12 outside of the boundary 30 of the suction portion 32 (i.e., at the additional portion 34) so as not to interfere with suction created by the suction portion. Such a string or cord may also be attached to the body 12 by way of a hole formed through an appendage (similar to the hole 88 through the appendage 82 shown in FIG. 7) such as a tab extending from the periphery 18 of the body. The writing instrument may also be connected to the device 12 by way of the item 40, such as by being attached to the pad of paper by way of the string or cord.

As described above, the device 10 may include a message 46 formed on or in the body 12. Messages 46 formed in the body 12 may be formed at an intermediate position between the outer surface 14 and the inner surface 16 of the body. In the embodiment illustrated in FIG. 1, the message 46 includes advertising information for a tree farm or nursery displayed on the outer surface 14 of the body 12. Various types of messages 46 may be displayed on or in the flexible body 12 without departing from the scope of the present invention. For example, words, logos, and/or art may be displayed on or in the flexible body 12 for communication and/or aesthetic purposes. The message 46 may be secured to or formed on or in the body 12 in various ways without departing from the scope of the present invention. For example, the message 46 may be formed on the outer surface 14, on the inner surface 16, and/or between the inner surface and the outer surface of the body 12. As a particular example, the message 46 may be printed on the outer surface 14 or on the inner surface 16 of the body 12. The message 46 may also be printed on a label adhered to the outer surface 14 and the label may be fully or partially opaque, translucent, and/or transparent as desired. It is contemplated that the message 46 may be formed using material of the flexible body 12 by, for example, molding, melting, etching, engraving, doping, or impressing the message into the outer surface 14 of the body.

The aroma of the scented body material may correspond to the message 46 displayed on or in the body 12. Further, the message 46 may be colored as desired and the shape of the body 12, the color of the body, the message, the color of the message, and the aroma emitted by the body may be selected to relate to each other as desired. For example, in an embodiment (not shown) similar to an embodiment described above, the body 12 is shaped as an ice cream cone and includes a light brown color at a cone portion of the cone and a pink color at an ice cream portion. The body 12 of this embodiment may also include accents forming waffle lines (e.g., in three-dimension) on the cone and details of the ice cream (e.g., raised and lowered portions forming scoops of ice cream on the cone) and a message 46 providing information about, for example, an ice cream store or an ice cream social sponsored by a church formed (e.g., by dark pink printing) on the outer surface of the body in the cone portion. The body 12 of this embodiment may further include material emitting a strawberry aroma corresponding to the ice cream represented by the shape of the periphery, the coloring of the ice cream, and the coloring of the message reflecting strawberry ice cream. As another example (not shown), the body 12 may include the name of a candy store (e.g., printed on the outer surface 14) and the body may include a material emitting a candy-type aroma, such as cinnamon or mint. As yet another example, the body 12 may include the business and/or domain name of a florist (see for example, the message 156 formed on the body 132 in FIG. 9) and a material emitting a floral aroma.

As described above, the device 10 may include markings and/or accents (exemplary markings 154, 190, 210 shown in FIGS. 9, 11, and 12), such as raised and/or lowered portions of material formed on the outer surface 14 of the body 12 thereby providing the device with more of a three-dimensional appearance. Material forming the accents may be unitary with material forming the body 12 or separate from and attached to the body material. As an example of a raised accent, in one embodiment (not shown) the body 12 has a periphery 18 shaped as a dolphin and includes raised and/or lowered portions forming portions (e.g., a rounded body, fins, flippers, flukes, eye sockets, and a blow hole) of the dolphin. Further in this embodiment, the body forming the dolphin shape may be colored blue and include markings (e.g., black printed lines) forming details of the dolphin, such as details of the body, an eye, and the blow hole. The three-dimensional accents of the body 12 may correspond to the aroma emitted by the body. For example, a body 12 having a periphery 18 shaped as a head of a flower (such as the bodies 86, 132 shown in FIGS. 7 and 9) and including raised and/or lowered accent portions and markings (such as the markings 154 in FIG. 9) forming details of the flower may also include material emitting a floral aroma.

FIG. 6 shows a device 50 according to an embodiment of the present invention including a body 52 having an outer surface 54, an inner surface 56 opposite the outer surface, and a periphery 58 extending around the body and surrounding the inner surface and the outer surface. The periphery 58 of the body 52 may have various shapes including those described above regarding the body 12 of the first embodiment. The body 52 of the device 50 shown in FIG. 6 is shaped as a heart (e.g., a cardiod). The body 52 includes material (not shown in detail) emitting a desired scent or aroma such as those described above regarding other embodiments. The outer surface 54 may include a convex portion 60. The inner surface 56 includes a concave portion 62 opposite the outer surface 54. For embodiments in which the outer surface 54 includes the convex portion 60, the concave portion 62 of the inner surface 56 may be opposite the convex portion. The inner surface 56 of the body 52 may further include a suction portion 64 surrounded by a boundary 66. The suction portion 64 consists of the surface of the body 52 that interacts with the mounting surface 26 to create the suction mounting the device 50 on the mounting surface. The suction portion 64 includes the concave portion 62 of the inner surface 56 and the boundary 66 extends around and defines the suction portion. The body 52 further includes an additional portion 68 between the suction portion 64 and the periphery 58. The additional portion 68 of the body 52 is not needed to create the suction used to mount the device 50 on the mounting surface 26.

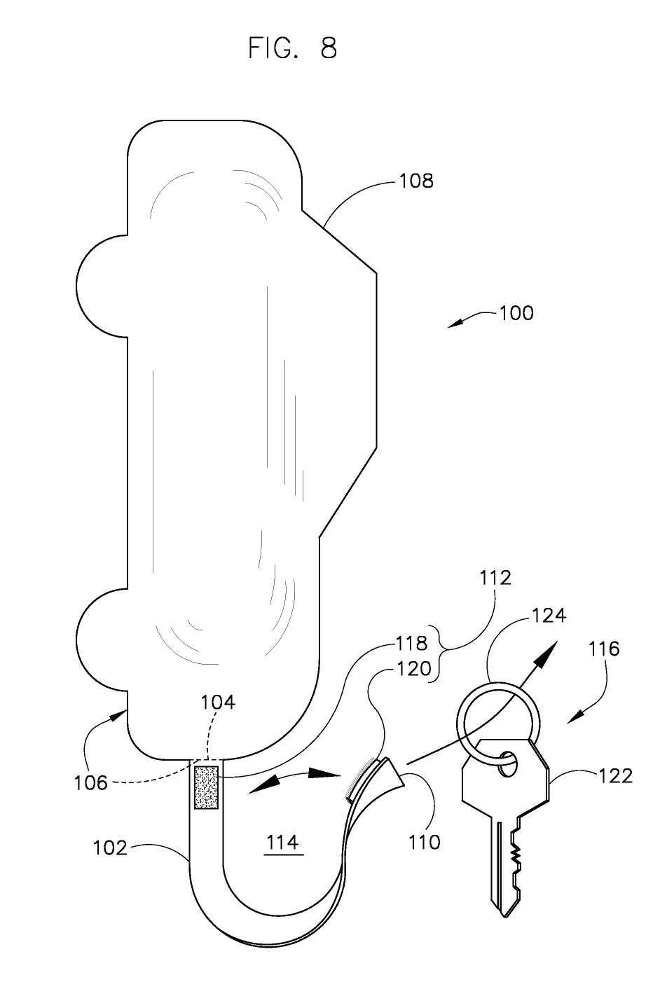

The device 50 according to this embodiment includes a hole 70 through the body 52 for holding a string or cord. The hole 70 may also be used to connect an object (such as the object 116 in FIG. 8) such as one of the items described above to the body 52. The object may be connected to the body 52 directly or by way of a connector or link such as a conventional key chain link. The object may include the link or connector. The device 50 may include various types of links without departing from the scope of the present invention. In one embodiment, the link includes a ring (such as the ring 124 in FIG. 8), such as a conventional key ring. As another example, the link may include a flexible member, such as a linked chain, a nylon, plastic, or metal cord, or a flexible strap, connected to the body 52 through the hole 70 for holding the object during use of the device 50.

The device 50 may be used to hold a variety of objects without departing from the scope of the present invention. For example, the device 50 may hold a key (such as the key 122 shown in FIG. 8), a flashlight, cards such as a credit or an identification card, a pager, a cellular telephone or personal data assistant, a picture frame, a charm, a calendar, designs and/or communications such as advertisements encased in plastic or lamination, or a clip such as those that can be releasably connected to a belt loop.

As described above, any hole 70 extending through the body 52 must extend through the body at the additional portion 68 of the body so as not to interfere with suction created by the suction portion 64. Further, whether the hole is formed through the body 52 or an appendage (such as the appendage 82 shown in FIG. 7) extending from the periphery 58 of the body, the device 50 must be configured so there is sufficient material for holding the object. If the additional portion 68 having the hole 70 is too short relative to the object and/or link, the object and link will interfere with mounting the device 50 on the mounting surface 26 or may undesirably demount the device after it had been secured to the mounting surface by pushing the device away from it.

The aroma emitted by the body 52 may correspond to the shape or other quality of the object attached to the body. In one embodiment, the aroma corresponds to a message 72 printed on the object. For example, the scented material of the body 52 may emit a floral aroma and the object may be shaped as a flower and/or include floral details or a florist's contact information printed on the object. The aroma corresponding to the object may also correspond to other characteristics of the device 50 such as the shape of the body 52 and a message 72 displayed on and/or in the body. In FIG. 6, the message 72 includes the word “LOVE” wherein the “O” is part of a flower design. The body 52 of this embodiment may include a material emitting an aroma relating to the message 72 formed on and/or in the body, such as a floral scent relating to the flower part of the message. In another embodiment (not shown), an object colored, shaped, and marked as an orange is attached to the body 52 that is shaped as a heart, includes the words “I LOVE ORANGES” printed on the outer surface 54 of the body, and includes material emitting an orange or citrus aroma. In yet another embodiment (not shown in detail), the object is shaped as a pineapple and the body 52 includes material emitting a pineapple aroma. In still another embodiment (not shown in detail), the object attached to the body 52 displays information about a chocolatier, such as the company's logo and domain name, and the body includes material emitting a chocolate aroma. The device 50 according to this embodiment may otherwise include characteristics of the device 10 described above and therefore will not be described in further detail.

FIG. 7 shows a device 80 according to an embodiment of the present invention including an appendage 82 extending from a periphery 84 of a body 86 including material emitting a desired aroma. The appendage 82 may include a hole 88 for connecting a string, cord, or an object to the body 86. The object may be any of the objects described above regarding other embodiments. The object may be connected to the appendage 82 directly or by way of a connector or link (such as the ring 124 shown in FIG. 8) such as a conventional key chain link. The device 80 may also include a fastener (such as the snap or fastener 38 shown in FIG. 5) connected to the appendage 82. The device 80 further includes markings or accents 92 formed on or in the body 86.

FIG. 8 shows a device 100 including an appendage 102 having a base 104 connected to a periphery 106 of a body 108 and a tip 110 opposite the base. The body 108 includes material emitting a desired aroma such as those described above regarding other embodiments. The appendage 102 of this embodiment may include a fastener 112 for releasably or permanently connecting the tip 110 of the appendage to the body 108 or the appendage adjacent the base 104 of the appendage thereby forming a loop 114 for holding an object or item 116. The device 100 may include various types of fasteners 112 for attaching an item to the device. Exemplary fasteners 112 include hook-and-loop, snap, adhesive, magnet, and latch. The fastener 112 may permanently connect the appendage 102 adjacent the tip 110 to itself or to the body adjacent the base 104, such as by adhering, stapling, or fusing (e.g., by melting) the appendage adjacent the tip to the appendage or body adjacent the base.

The object 116 may be selected from the objects and items described above regarding other embodiments. The object 116 shown in FIG. 8 is a key 122 and a chain or link 124. The link 124 may be part of the device 100. The device 100 must be configured so that when an object 116 is attached to the loop 114 formed by the appendage 102, the loop and object do not interfere with suction created by a suction portion (similar to the suction portions 32, 64 described above) of the body 108. If the appendage 102 is too short relative to the object 116 and any link 124 connecting it to the device 100, the object and/or link will interfere with mounting the device on the mounting surface 26 or may undesirably demount the device after it had been secured to the mounting surface by pushing the device away from it. The device 100 according to this embodiment may otherwise be the same as the devices 10, 50, 80 described above and therefore will not be described in further detail.

As described above, the suction-mountable devices according to the present invention may also include a protrusion. FIG. 9 shows an embodiment of a device 130 including a flexible or deformable body 132 having an outer surface 134, an inner surface 136 opposite the outer surface, and a periphery 138 extending around the body and surrounding the inner surface and the outer surface. The body 132 includes material emitting a desired aroma such as those described above regarding other embodiments. The outer surface 134 may include a convex portion 140. The inner surface 136 includes a concave portion 142 opposite the outer surface 134. For embodiments in which the outer surface 134 includes the convex portion 140, the concave portion 142 of the inner surface 136 may be opposite the convex portion.

The device 130 according to this embodiment includes a protrusion 144 extending from the outer surface 134 of the body 132. The protrusion 144 and the body 132 may be unitary (e.g., formed as one piece) or separate connected parts. The protrusion 144 includes a head 146 having an outer or exterior surface 148 and a periphery 150 extending around the protrusion and surrounding the exterior surface of the head. The head 146 may be connected directly to the body 132 or the protrusion 144 may include a stem 152 connecting the head to the outer surface 134 of the body. The periphery 150 of the protrusion 144 may have various shapes without departing from the scope of the present invention including those described above regarding the body peripheries 18, 58, 84, 106 of other embodiments of the present invention. For example, the periphery 150 of the protrusion 144 may be non-circular, symmetric, such as circular, square, or oval, or non-symmetric. Also, the protrusion 144 may be colored or tinted as desired, such as described above regarding the bodies 10, 50, 80, 100 of other embodiments of the invention.

The protrusion 144 may include material emitting an aroma along with or instead of the body 132 including material emitting an aroma. The scent of the body 132 and/or the protrusion 144 may correspond to any one or combination of the color of the protrusion and/or the body, the shape of the protrusion and/or the body, and markings, accents, or messages formed on the protrusion and/or the body. The markings, accents, colors, and messages may be the same as any of the markings, accents, and messages described above regarding other embodiments. In the embodiment shown in FIG. 9, the body 132 is shaped, colored, marked, and/or accented as the head of a flower and the protrusion 144 is shaped, colored, marked, and/or accented as a butterfly. The markings 154 include details of the flower head formed on the body 132 and details of the butterfly formed on the protrusion 144. The device 130 further includes a message 156 of a florist's business name and/or contact information such as web address (i.e., internet domain name) formed on or in the body 132. In this embodiment, the scented material emits a floral aroma corresponding to the flower shape of the body 132, the butterfly shape of the protrusion 144, the markings 154 forming details of the flower, the markings forming details of the butterfly, and the message formed on or in the body relating to the florist. The floral aroma relates to butterflies because butterflies often land on flowers in nature.

The protrusion 144 may form three-dimensional objects by including raised and/or lowered accents similar to the manner in which the bodies described above regarding other embodiments form three-dimensional objects. For example, in one particular embodiment (not shown) the head 146 forms a three-dimensional baseball by being generally spherical (i.e., including a raised protrusion forming a sphere) and colored white and including additional raised portions colored red forming stitching of the ball. As another example (not shown), a head 146 having a periphery 150 shaped as a dolphin may also include raised and/or lowered portions forming parts (e.g., a rounded body, fins, flippers, flukes, eye sockets, and a blow hole) of the dolphin. Further in this embodiment, the head 146 forming the dolphin shape may be colored blue and include markings (e.g., black printed lines) forming details of the dolphin, such as details of the body, an eye, and the blow hole. The aroma emitted by the body 132 may correspond to the accents formed on or by the protrusion 144. For example, a protrusion 144 having a periphery 150 shaped as a pizza can include material emitting a pizza-like aroma (e.g., a scent of cheese and pizza sauce).

FIG. 10 illustrates a device 160 including a body 162 and a protrusion 164 having messages 166 including the letter “I” displayed on an exterior surface 168 of the head 170 of the protrusion and the words “LOVE” and “COFFEE” displayed on the outer surface 172 of the body. In this embodiment, the body 162 and the protrusion 164 are shaped as hearts and the body and/or the protrusion emit a coffee aroma corresponding to the message 166 displayed on the outer surfaces 168, 172 of the protrusion and the body. As an example of the aroma corresponding to the shape of the peripheries 174, 176 of the body 162 and the protrusion 164, the device may be the same as the immediately preceding example (i.e., having a heart-shaped body and protrusion and emitting a coffee aroma), except that the body is shaped as a coffee cup and the protrusion is shaped as a heart. Alternatively, the body 162 and/or the protrusion 164 may emit a coffee aroma and be shaped as a heart and the protrusion may be shaped as a coffee cup.

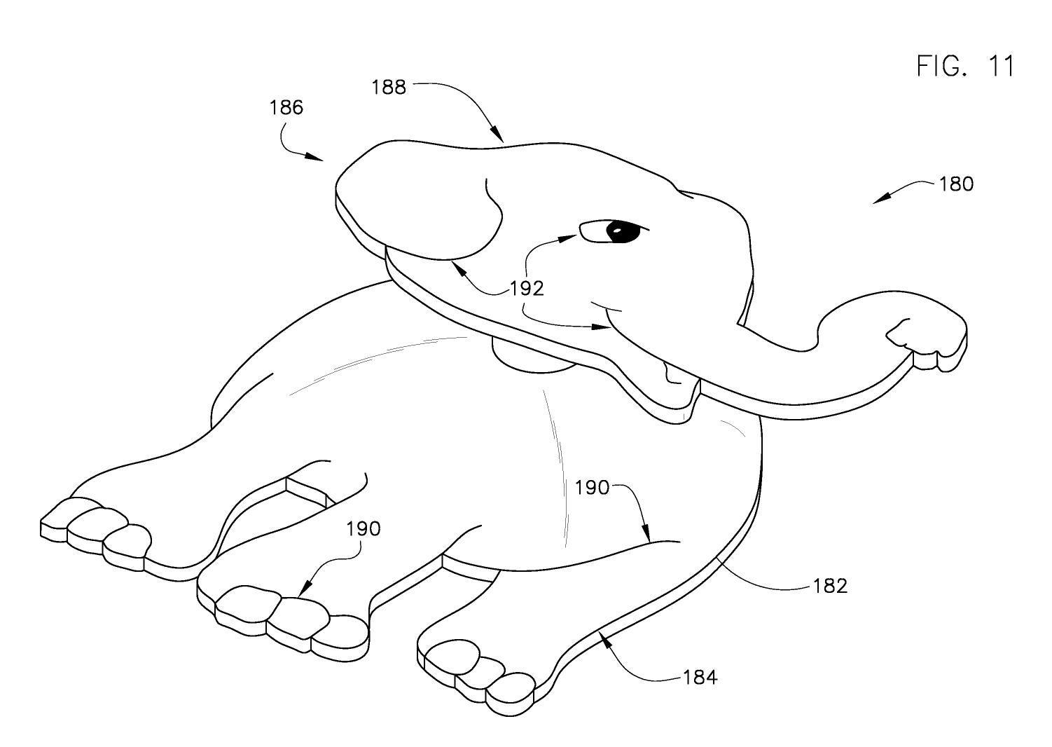

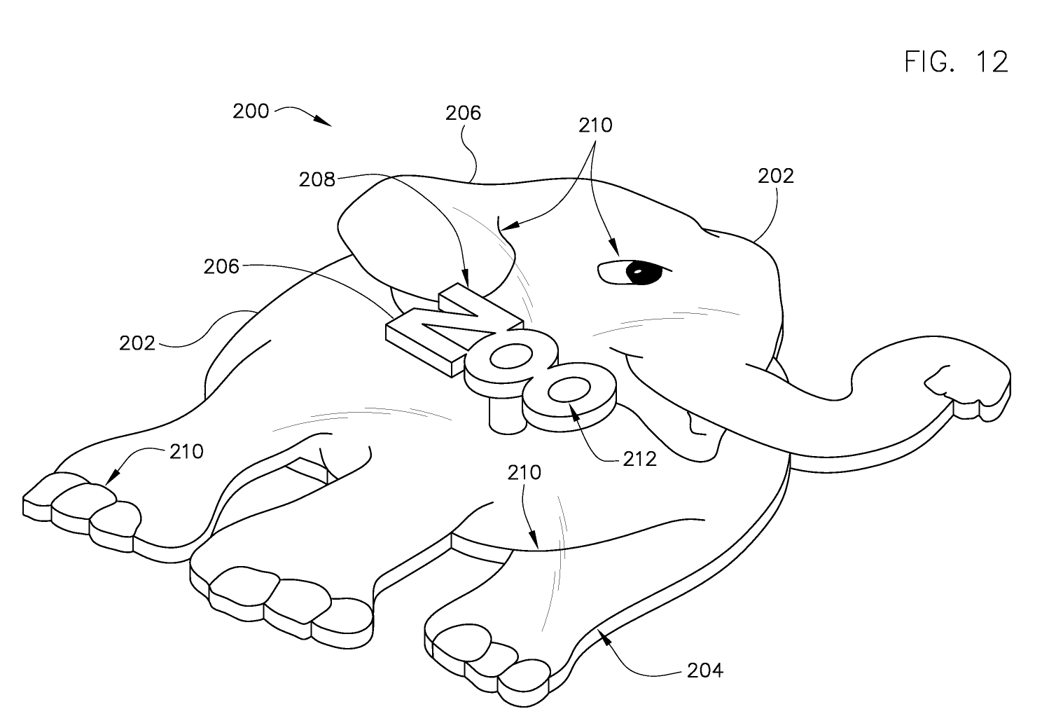

The shape of the periphery of the protrusion and the shape of the periphery of the body may be generally the same. For example, the peripheries of the body and the protrusion are both heart shaped. The same-shaped peripheries may be oriented in the same way (i.e., same direction) or differently (i.e., angularly offset from each other). In some embodiments, the shape of the periphery of the protrusion is different from the shape of the periphery of the body (see e.g., FIGS. 11 and 12). The shapes of the peripheries of the body and the protrusion may correspond to each other and the aroma emitted by the scented material of the body may correspond to one or both periphery shapes. That is, the periphery of the protrusion may be shaped as an item that is related to an item that the periphery of the body is shaped as and the scented material of the body may correspond to one or both periphery shapes. One example of the peripheries corresponding to each other is the peripheries having the same shape.

Differently shaped peripheries of a body and a protrusion may also relate to each other. For example, FIG. 11 shows a device including a body having a periphery shaped as a body of an elephant and a protrusion having a periphery shaped as an elephant head corresponding to the elephant body. Such a device may be used, for example, as a marketing device for a safari tour company. As shown in FIG. 11, the body and protrusion include markings forming details of the elephant body and head, respectively.

As another example of differently shaped bodies and protrusions relating to each other, FIG. 12 shows a device including a body having a periphery shaped as an elephant (including the body and the head of the elephant) and a protrusion having a periphery shaped as letters forming the word “ZOO”. As shown in FIG. 12, the body and the protrusion include markings forming details of the elephant and the wording, respectively. Such as device 200 may be used for, for example, marketing a public zoo. The shape of the protrusion 206, forming the word “ZOO”, relates to the elephant-shaped body 202 because elephants are common animals at zoos. As yet another example of a device having related body and protrusion shapes, in one particular embodiment (not shown) the periphery of the body is shaped, colored, and marked, and/or accented as a football helmet and the periphery of the protrusion is shaped and colored, marked and/or accented as a football. In another embodiment (not shown), the periphery of the body is shaped and colored, marked, and/or accented as a baseball hat and the periphery of the protrusion is shaped and colored, marked, and/or accented as a baseball and/or a baseball bat.

The shape of the protrusions may communicate a message or theme similar to the manner that the shape of the body 12 of the device 10 according to the first embodiment (shown in FIG. 1) form shapes communicating messages and/or themes as described above. Further, the peripheries of the body and the protrusion may communicate the same, related, or unrelated messages or themes. For example, in one embodiment (not shown) the peripheries of the body and the protrusion are shaped as arrows and the arrows point in the same direction (i.e., the peripheries have the same orientation). This arrow-shaped device can be used to indicate a particular direction or to point to an item. For example, an arrow-shaped device including material that glows in the dark can be secured to a wall adjacent and pointing to a door in a darkened room such as a movie theater or an x-ray room. In this example, the arrow-shaped device would indicate a location of the door or doorknob. Also, the peripheries of the body and the protrusion may operate together to create a message or theme. For example, the periphery of the body may be shaped as a cigarette and the periphery of the protrusion may be shaped as a circle having a bar through it colored red indicating that smoking is prohibited in the area where the device is mounted. As yet another example, the periphery of the body may be shaped as an arrow and the periphery of the protrusion may be shaped as the circle having the bar through it colored red indicating that the viewer of the device should not go in a direction that the arrow-shaped body is pointing.

Another example of the peripheries of the body and/or protrusion forming messages or themes is the peripheries being shaped as letters, numbers, a logo, or other symbols or characters. The protrusion 206 shown in FIG. 12 form letters forming the word “ZOO”. As another example, the body may be shaped, colored, marked, and/or accented (e.g., textured) as a smiling face and the protrusion may be shaped to form the letters “HELLO”. As another example, the body may be shaped and marked and/or accented a hand with the first two fingers forming a V shape (i.e., the popular manual sign for peace) and the protrusion may be shaped and marked to form the word “PEACE”. As yet another example, the body may be shaped and marked and/or accented as a heart (like the body 132 shown in FIG. 9) and the protrusion may be shaped to form as letters forming the word “LOVE”. The periphery of the body and/or the periphery of a protrusion may form messages by forming words relating to the aroma emitted by the scented material of the body. For example, the body and/or the protrusion may form a word such as a name of a fruit or flower and the body may include material emitting the corresponding aroma (i.e., a fruit scent or floral scent, respectively).

In one embodiment, the peripheries of the body and/or the protrusion have shapes relating to an entity using the device, such as a store or restaurant and the scented material of the body may relate to the entity. For example, the peripheries of the body and/or the protrusion may be shaped as apples or bananas and used at a fruit market and/or as an advertisement for the market. The peripheries of the body and the protrusion may have different shapes relating to the same message or theme. For example, in one embodiment (not shown) the periphery of the body is shaped as an apple and the periphery of the protrusion is shaped as another fruit, such as a pineapple. The fruit-shaped peripheries of these examples may communicate, among other things, that fruit is sold in the area where the devices are mounted. As another example, the periphery of the body may be shaped as an apple and the periphery of the protrusion may be shaped as a fish to indicate that produce and seafood are sold in the area where the device is mounted. The particular periphery shapes relating to an entity described herein are only exemplary and the device may be designed to include peripheries having shapes relating to various other entities in various ways.

The exemplary periphery shapes described herein are not exclusive to the category that they are described with respect to. Accordingly, the examples provided in each category of the peripheries of the body and the protrusion corresponding to each other, communicating messages, relating to an entity, and communicating a theme of a company or person, may apply to each other. For example, although an example of a device including a body shaped as a flower and a protrusion forming the word “FLORIST” qualifies for the category of the peripheries of the protrusion and the body corresponding to each other, this exemplary device also fits within the categories of devices having a body and a protrusion having different periphery shapes, having periphery shapes communicating messages, having periphery shapes relating to an entity, and having periphery shapes communicating a theme of a company or person. When introducing elements of the present invention or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A deformable aromatic device mountable by suction on a generally smooth mounting surface comprising:
a body including a flexible base formed from a first material and having an outer surface, an inner surface including a concave portion opposite the outer surface, a periphery extending around the body, surrounding the outer surface and the inner surface of the body, and a message formed on the body at a position selected from a group of positions consisting of an outer position on the outer surface, an inner position on the inner surface, and an intermediate position between the outer surface and the inner surface; and
a second material selected to emit a desired aroma connected to the base;
wherein the body is deformable from an undeformed shape to an actuated shape to mount the body on the mounting surface by positioning the inner surface of the body adjacent the mounting surface when the body is in the undeformed shape thereby forming a cavity between said concave portion and the mounting surface and deforming the body to its actuated shape thereby sealing the body against the mounting surface and forcing fluid out of the cavity to create suction between the body and the mounting surface thereby securing the body to the mounting surface.

2. A device as set forth in claim 1 wherein the message includes at least one character of a group of characters consisting of a letter and a number.

3. A device as set forth in claim 1 wherein the message is printed on the outer surface of the body, printed on the inner surface of the body, printed on a label adhered to the outer surface of the body, molded into the body, melted into the body, etched into the body, doped into the body, impressed into the body, or engraved into the body.

4. A device as set forth in claim 1 wherein the aroma corresponds to the message formed on the outer surface, on the inner surface, and/or between the outer surface and the inner surface of the body.

5. A device as set forth in claim 1 wherein the periphery of the body is non-circular.

6. A device as set forth in claim 1 wherein the aroma corresponds to a shape of the periphery of the body.

7. A device as set forth in claim 1 wherein said outer surface of the body is substantially free of protrusions.

8. A device as set forth in claim 1 wherein the body includes a color corresponding to said aroma.

9. A device as set forth in claim 1 wherein said second material emitting the desired aroma is impregnated into said first material.

10. A device as set forth in claim 1 wherein said second material emitting the desired aroma is coated on at least one surface of a group of surfaces consisting of the outer surface of the body and the inner surface of the body.

11. A device as set forth in claim 1 further comprising a protrusion extending from the outer surface of the body.

12. A deformable aromatic device mountable by suction on a generally smooth mounting surface comprising:
a body including a flexible base formed from a first material and having an outer surface, an inner surface including a concave portion opposite the outer surface, and a periphery extending around the body, surrounding the outer surface and the inner surface of the body; and
a protrusion extending from the outer surface of the body, the protrusion including a second material emitting a desired aroma;
wherein the body is deformable from an undeformed shape to an actuated shape to mount the body on the mounting surface by positioning the inner surface of the body adjacent the mounting surface when the body is in the undeformed shape thereby forming a cavity between said concave portion and the mounting surface and deforming the body to its actuated shape thereby sealing the body against the mounting surface and forcing fluid out of the cavity to create suction between the body and the mounting surface thereby securing the body to the mounting surface.

13. A deformable aromatic device mountable by suction on a generally smooth mounting surface comprising:
a body including a flexible base formed from a first material and having an outer surface, an inner surface including a concave portion opposite the outer surface, and a periphery extending around the body, surrounding the outer surface and the inner surface of the body; and
a second material selected to emit a desired aroma connected to the base, said aroma corresponding to a shape of the periphery of the body;
wherein the body is deformable from an undeformed shape to an actuated shape to mount the body on the mounting surface by positioning the inner surface of the body adjacent the mounting surface when the body is in the undeformed shape thereby forming a cavity between said concave portion and the mounting surface and deforming the body to its actuated shape thereby sealing the body against the mounting surface and forcing fluid out of the cavity to create suction between the body and the mounting surface thereby securing the body to the mounting surface.

14. A device as set forth in claim 13 wherein the periphery of the body is non-circular.

15. A device as set forth in claim 13 wherein the body includes at least one color corresponding to said aroma.

16. A deformable aromatic device mountable by suction on a generally smooth mounting surface comprising:
a body including a flexible base formed from a first material and having an outer surface, an inner surface including a concave portion opposite the outer surface, and a periphery extending around the body, surrounding the outer surface and the inner surface of the body;
a protrusion extending from the outer surface of the body including an exterior surface and a periphery extending around the protrusion and surrounding the exterior surface; and
a second material selected to emit a desired aroma connected to the base, said aroma corresponding to a shape of the periphery of the protrusion;
wherein the body is deformable from an undeformed shape to an actuated shape to mount the body on the mounting surface by positioning the inner surface of the body adjacent the mounting surface when the body is in the undeformed shape thereby forming a cavity between said concave portion and the mounting surface and deforming the body to its actuated shape thereby sealing the body against the mounting surface and forcing fluid out of the cavity to create suction between the body and the mounting surface thereby securing the body to the mounting surface.

17. A deformable aromatic device mountable by suction on a generally smooth mounting surface comprising:
a body including a flexible base formed from a first material and having an outer surface, an inner surface including a concave portion opposite the outer surface, and a non-circular periphery extending around the body, surrounding the outer surface and the inner surface of the body; and
a second material selected to emit a desired aroma connected to the base, said aroma corresponding to a shape of the periphery of the body;
wherein the body is deformable from an undeformed shape to an actuated shape to mount the body on the mounting surface by positioning the inner surface of the body adjacent the mounting surface when the body is in the undeformed shape thereby forming a cavity between said concave portion and the mounting surface and deforming the body to its actuated shape thereby sealing the body against the mounting surface and forcing fluid out of the cavity to create suction between the body and the mounting surface thereby securing the body to the mounting surface.

18. A deformable aromatic device mountable by suction on a generally smooth mounting surface comprising:
a body including a flexible base formed from a first material and having an outer surface, an inner surface including a concave portion opposite the outer surface, and a periphery extending around the body, surrounding the outer surface and the inner surface of the body;
a second material selected to emit a desired aroma connected to the base, said aroma corresponding to a shape of the periphery of the body; and
a fastener attached to the outer surface of the body for attaching an item to the device;
wherein the body is deformable from an undeformed shape to an actuated shape to mount the body on the mounting surface by positioning the inner surface of the body adjacent the mounting surface when the body is in the undeformed shape thereby forming a cavity between said concave portion and the mounting surface and deforming the body to its actuated shape thereby sealing the body against the mounting surface and forcing fluid out of the cavity to create suction between the body and the mounting surface thereby securing the body to the mounting surface.

19. A device as set forth in claim 18 wherein the second material emitting the aroma corresponds to a shape of the item and/or to a message formed on the item.

20. A device as set forth in claim 18 wherein the fastener includes an adhesive or a part of a hook-and-loop fastener.

21. A method of using an aromatic device including a deformable body having an outer surface, an inner surface having a concave portion opposite said outer surface, a periphery extending around the body and surrounding the inner surface and the outer surface, and a material emitting a desired aroma, the method comprising:
positioning the inner surface of the body adjacent a mounting surface in an area occupied by people or used by people to store things so the aroma emitted from the body may be sensed by the people when they are in or near the area, the inner surface of the body being positioned adjacent the mounting surface while the body is in an undeformed shape thereby forming a cavity between the body and the mounting surface; and
moving the body to an actuated shape thereby sealing the body against the mounting surface and forcing fluid out of the cavity to create suction between the body and the mounting surface thereby securing the body to the mounting surface in said area.

22. A method of using an aromatic device as set forth in claim 21 wherein the positioning step includes positioning the inner surface of the body adjacent a particular mounting surface in said area selected from a group of particular mounting surfaces consisting of an interior surface of an automobile, a surface of a locker, a wall, a window, a mirror, a component of a computer, and a desk, and the moving step includes securing the body to the particular mounting surface in said area.
